(19) United States
(12) Patent Application Publication
(10) Pub. No.: US 2012/0071858 A1
(43) Pub. Date: Mar. 22, 2012

(54) Vaginal therapeutic device including copper metal and method of treating the vagina using the vaginal therapeutic device

(76) Inventors: Chun Lim Abbott, Pittsburgh, PA (US); Dominic Abbott, Pittsburgh, PA (US)

(21) Appl. No.: 13/317,230

(22) Filed: Oct. 12, 2011

Related U.S. Application Data
(63) Continuation of application No. 12/157,823, filed on Jun. 13, 2008, now abandoned.

(51) Int. Cl. A61M 31/00

(52) U.S. Cl. 604/515; 604/285

(57) ABSTRACT

A vaginal therapeutic device made of copper metal has a shape to fit in the vagina and utilizes the copper metal as an antimicrobial agent to therapeutically treat abnormal biological conditions in the vagina.

### BACKGROUND OF THE INVENTION

1. Field of the Invention

The present invention relates to therapeutic treatment of abnormal conditions in the vagina caused by microbial organisms and, more particularly, to such treatment utilizing a vaginal therapeutic device having an exterior surface made of copper metal.

2. Brief Discussion of the Related Art

In the area of female personal hygiene and gynecological health, many efforts have been made to reduce microbial organisms in the vagina which create abnormal conditions such as vaginal odor, bacterial and viral infections, yeast infections and the like. Most of the attempts have involved medication and/or douching. Particularly effective douching systems utilizing a stainless steel douche appliance and methods using metal oxides are described in U.S. Pat. No. 6,190,365, U.S. Pat. No. 6,589,216, U.S. Pat. No.7,276,056 and U.S. Pat. No. 7,270,653 to Abbott et al. Abnormal vaginal conditions are caused by various microbial organisms including bacterial, viral and fungal organisms. No device or associated method has been found to provide an anti-microbial treatment to kill such microbial organisms.

It is known that many bacteria known to be human pathogens cannot survive on surfaces of copper metal. As used herein, “copper metal” means pure copper and copper alloys such as brasses, bronzes, copper-nickels and copper-nickel-zincs as described in “Abstract from Copper Alloys for Human Infection Disease Control” by H. T. Michaels, S. A. Wilks, J. O. Noyce and C. W. Keivel. The number of live bacteria drops from several orders of magnitude to almost zero on copper metal in a few hours. Copper metals which have been tested include high (pure) coppers, brasses, bronzes, copper-nickels, and copper-nickel-zincs. The bacteria tested include Methicillin-resistant staphylococcus aureus (MRSA), the cause of serious hospital-acquired infections, pseudomonas aeruginosa enterbacter aerogens and Acinetobartor baumonaii, as well as E. coli 0157:H7 and listeria monocytogenes, foodborne pathogens associated with several large-scale food recalls. Copper metal as a “static” agent inhibits microbial growth by means other than killing such that it limits the growth of microorganisms and may inactivate them. Copper metal as an “antimicrobial” substance (chemical or physical) can prevent microbial growth either by some ‘-static’ action or by the outright killing of microbial organisms. Copper metal as a “-cidal” agent either damages a microbial organism at low concentration and/or reduced contact time or interacts permanently so that it ceases to function normally. Such “-cidal” agent damages a microorganism sub-lethally. Total inactivation is functionally equivalent to killing the organism (0% survival).

### SUMMARY OF THE INVENTION

In accordance with the present invention, a copper metal surface is provided in the vagina for a sufficient period of time such that microbial organisms in the vagina are killed. In this manner, various bacterial conditions, fungal conditions and viral conditions are treated.

In one aspect, the present invention utilizes a vaginal therapeutic device formed of a body having an exterior surface made of copper metal, the body having a shape to fit in the vagina such that the copper metal exterior surface contacts the inner walls of the vagina to act as an anti-microbial agent.

In another aspect, the present invention treats abnormal biological conditions in the vagina by inserting a therapeutic device having an exterior surface made of anti-microbial copper metal in the vagina, retaining the therapeutic device in the vagina for a time sufficient to kill microbial organisms and thereafter removing the therapeutic device from the vagina.

Other aspects and advantages of the present invention will become apparent from the following description of the preferred embodiments taken in conjunction with the accompanying drawings.

### BRIEF DESCRIPTION OF THE DRAWINGS

FIG. 1 is a perspective view of a vaginal therapeutic device according to the present invention.

FIG. 2 is an exploded perspective view of the vaginal therapeutic device of FIG. 1.

FIG. 3 is a sectional view of a vaginal therapeutic device according to the present invention positioned within the vagina.

FIG. 4 is an exploded sectional view of a modification of a vaginal therapeutic device according to the present invention.

FIG. 5 is a perspective view of another modification of a therapeutic vaginal device according to the present invention in a contracted state.

FIG. 6 is a perspective view of the vaginal therapeutic device of FIG. 5 in an expanded state.

FIG. 7 is an elevation of a further modification of a vaginal therapeutic device according to the present invention in a contracted state.

FIG. 8 is an elevation of the vaginal therapeutic device of FIG. 7 in an expanded state.

### DETAILED DESCRIPTION OF THE INVENTION

A vaginal therapeutic device 10 according to the present invention is shown in FIG. 1 and is formed of a rigid body 12 having a rounded shape, cylindrical in cross-section, with a rounded exterior surface made of copper metal. As noted above, “copper metal” as used herein means pure copper (99.5% or greater copper) and copper alloys such as brasses, bronzes, copper-nickels and copper-nickel-zincs. The body 12 has a distal end 14 with a semispherical shape and a proximal end 16 that is rounded and has a longitudinally extending neck 18 with an opening 20 therein. A retraction structure 22 is inserted in the opening to be carried at the proximal end 16. The body 12 can be hollow or solid, and the retraction structure 22 is designed such that it can be inserted in the opening 20 to facilitate insertion of the device in the vagina and removal of the device from the vagina. The retraction structure can be made of a light weight (as compared to copper metal) material which can also be flexible, such as plastic. The retraction structure 22 includes a pivotally movable ring 24 passing through a bore in a spherical end 26 at the proximal end of a stem 28 which has a spherical distal end 30 to be “snapped” into opening 20.

Due to the weight of copper metal, it is preferred that the body 12 is hollow; however, the body 12 can be solid if desired. Additionally, it is preferred that the copper metal be pure copper, i.e. 99.95% copper after processing; however, while pure copper has the greatest anti-microbial effect, various copper alloys as described above, can be utilized.

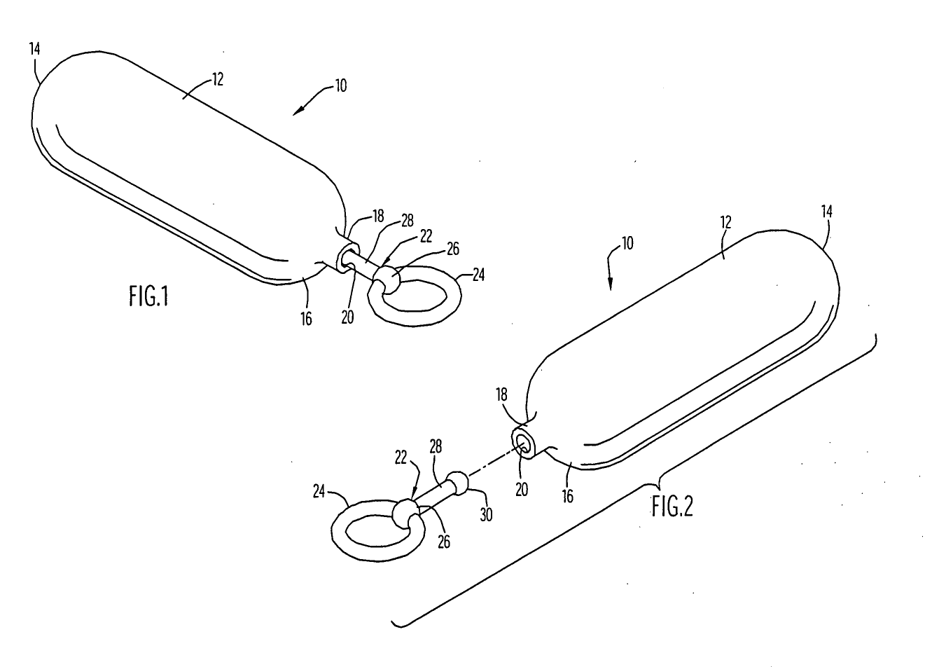

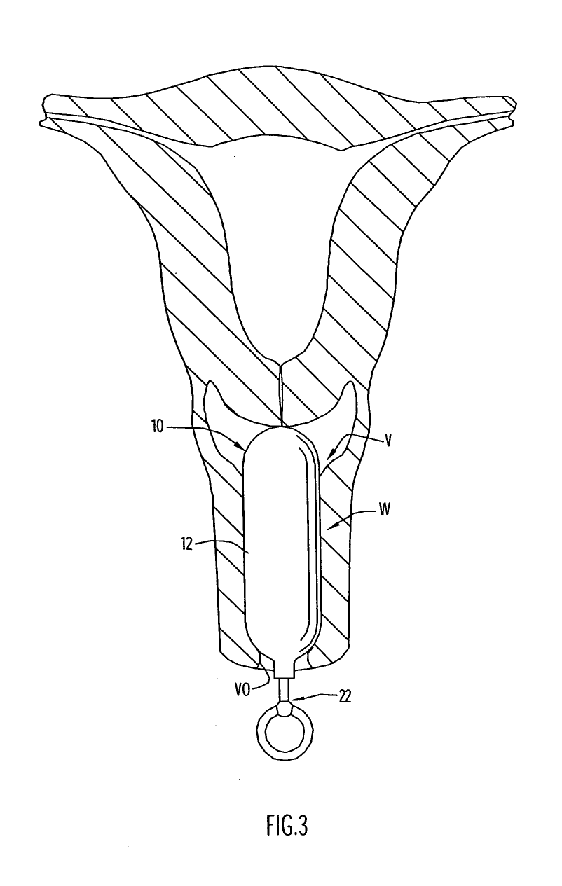

In use, the vaginal therapeutic device 10, inserted in the vagina V (vaginal canal) through the vaginal opening VO as shown in FIG. 3 with the retraction structure protruding therefrom, it being appreciated that the elastic nature of the walls W of the vagina will permit the inner walls of the vagina to contact the external surface 12 of the vaginal therapeutic device thereby causing microbial organisms to contact the copper metal. The time required to kill the microbial organisms depends upon the purity of the copper metal; however, if the device 10 is inserted in the vagina overnight, essentially all microbial organisms will be killed by the time the vaginal therapeutic device 10 is removed in the morning. The rounded shape of the body 12 facilitates insertion and removal of the vaginal therapeutic device in the rigid form shown in FIGS. 1 and 2.

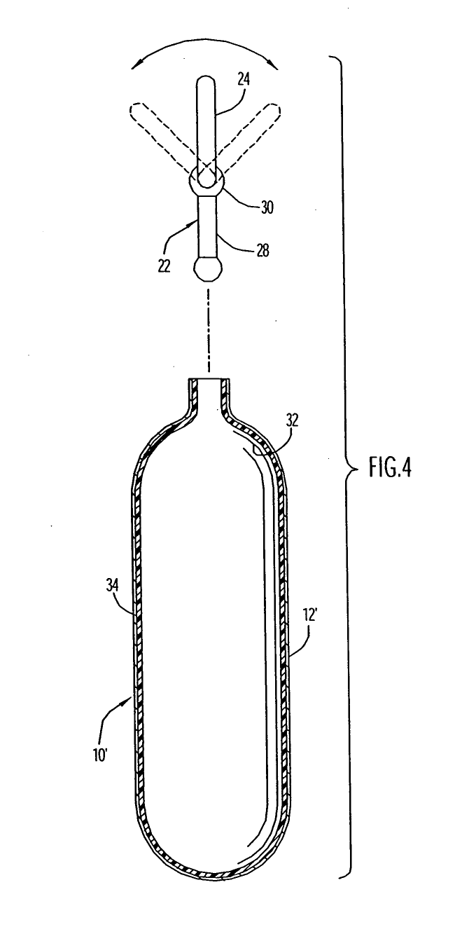

When the body 12 is hollow, the walls of the body can be solely copper metal. A lighter weight, less expensive modification of a vaginal therapeutic device 10?is shown in FIG. 4, wherein a body 12?is formed of an inner part 32 made of a less expensive, lighter weight material, such as plastic, with the inner part 32 carrying an outer layer 34 formed of copper metal.

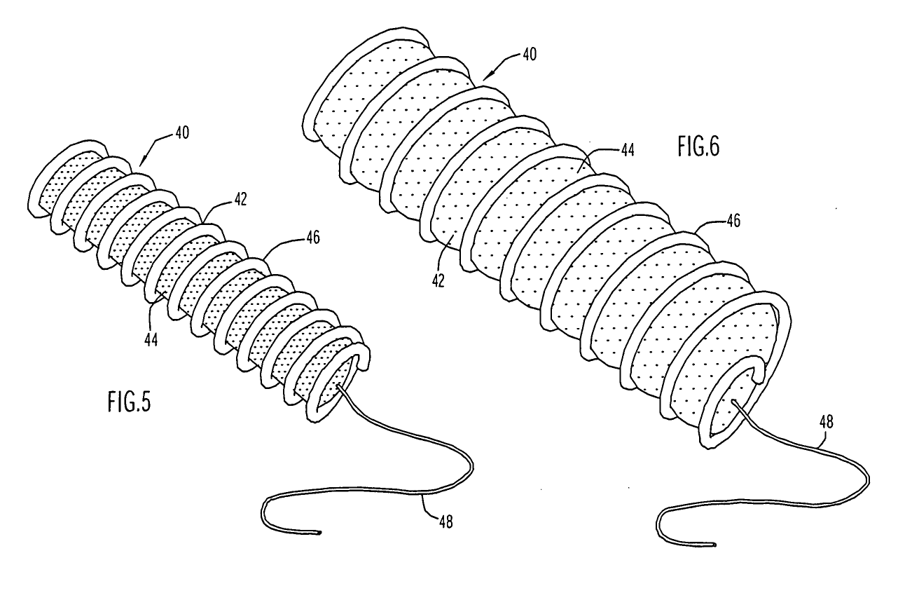

Another modification of a vaginal therapeutic device 40 according to the present invention is shown in a contracted state in FIG. 5 and has a body 42 including an expandable absorbent, sponge- or cotton-like material 44 having a rounded tampon-like shape with an expandable coiled wire 46 wrapped around the absorbent material, the coiled wire 46 being made of copper metal to form the exterior surface of the body for contacting microbial organisms in the vagina. Once the vaginal therapeutic device 40 is inserted in the vagina, the absorbent material 44 will expand from absorbing fluids within the vagina, and the coiled wire 46 will similarly expand, such that the vaginal therapeutic device 40 will have the expanded size shown in FIG. 6 to improve contact with the walls of the vagina. Of course, the vaginal therapeutic device 40 need not be expandable since the elasticity of the vaginal walls will provide good contact. A string 48 is attached to the proximal end of the body 42, in particular to the end of the absorbent material 44, to form a retraction structure for vaginal therapeutic device 40.

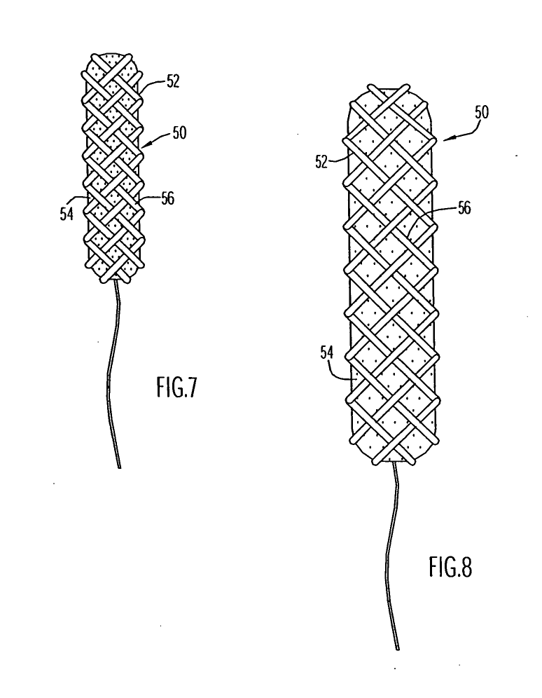

FIG. 7 shows another modification of a vaginal therapeutic device 50 according to the present invention having a body 52 formed of an expandable absorbent material 54 with a shape similar to the absorbent material of vaginal therapeutic device 40 but with an expandable mesh arrangement of wires 56 surrounding the absorbent material to form the exterior surface of the vaginal therapeutic device for contacting microbial organisms within the vagina, the wires of the mesh arrangement being made of copper metal to form the copper metal exterior surface of the vaginal therapeutic device. Once inserted, the absorbent material and the mesh arrangement of wires will expand as shown in FIG. 8 to facilitate contact of the external surface of the device with the walls of the vagina. Of course, the vaginal therapeutic device 50 need not be expandable since the elasticity of the vaginal walls will provide good contact. A string 58 is attached to the proximal end of the body 52 to form a retraction structure for vaginal therapeutic device 50.

To fabricate the vaginal therapeutic devices, it is noted that copper metal can be readily electro-formed, plated, hot rolled, extruded, cast or forged and can be cold rolled to a desired thickness to form sheets or thin layers or wire-like structures. The vaginal therapeutic device shown in FIG. 1 can, for example, be made of one piece or two or more welded pieces with a wall thickness of 0.06 mm with a length of 3.25 inches and a diameter of 0.75 inches. The ring of the retraction structure can, for example, have an outer diameter of 0.75 inches and an inner diameter of 0.625 inches.

When a vaginal therapeutic device according to the present invention is inserted or positioned in the vagina, the antimicrobial effect of the copper metal will kill or inactivate and inhibit growth of microbial organisms in the vagina which cause abnormal conditions, including viruses, bacterium, fungi and the like. Thusly, undesirable conditions such as odor, vaginitis, yeast infections, bacterial infections, viral infections, sexually transmitted diseases and the like are treated, cured or prevented, it being noted that HIV (human immunodeficiency virus), which causes AIDS, can be treated in accordance with the subject invention in that a vaginal eco-system imbalance caused by microbial organisms causes the vagina to be receptive to HIV.

Inasmuch as the present invention is subject to many variations, modifications and changes in detail, it is intended that all subject matter discussed above or shown in the accompanying drawings be interpreted as illustrative only and now be taken in a limiting sense.

1. A vaginal therapeutic device comprising
a rigid body having a rounded shape, cylindrical in cross-section with a rounded exterior surface made of copper metal, said body having a shape to fit and be retained in the vagina such that said copper metal exterior surface is retained in contact with the inner walls of the vagina whereby said copper metal acts as an antimicrobial agent to therapeutically treat abnormal biological conditions arising in and/or affecting the vagina.

2. A vaginal therapeutic device as recited in claim 1 wherein said body has opposed distal and proximal ends and further comprising a retraction structure carried at said proximal end of said body.

3.-4. (canceled)

5. A vaginal therapeutic device as recited in claim 1 wherein said body has a semi-spherical distal end and a proximal end with an opening therein and further comprising a retraction structure received in said opening in said proximal end.

6. A vaginal therapeutic device as recited in claim 1 wherein said body is formed of an inner part of a material other than copper melal and an outer layer formed of copper metal.

7. A vaginal therapeutic device as recited in claim 1 wherein said body is made entirely of copper metal.

8.-12. (canceled)

13. A vaginal therapeutic device as recited in claim 1 wherein said copper metal is a copper alloy.

14. A method of treating abnormal biological conditions in the vagina comprising the steps of
inserting a therapeutic device having an exterior surface made of antimicrobial copper metal in the vagina;
retaining the therapeutic device in the vagina with the exterior surface in contact with the inner walls of the vagina overnight for a time sufficient to kill microbial organisms; and
removing the therapeutic device from the vagina.

15. (canceled)

16. A vaginal therapeutic device as recited in claim 1 wherein said copper metal is pure copper.

17. A vaginal therapeutic device as recited in claim 16 wherein said body is hollow.
